SEQUENCE LISTING (3 sequences)

SEQ ID NO: 1 - DNA, 526 bp, synthetic construct
  source 1..526: mol_type = other DNA; note = Gene sequence of calmodulin of the Aspergillus flavus strain EXY1A109
gcgggatctatgccttcgagttagtatgctttggaccaaggaactcctcaaaagcatgatctcggatgtgtcctgttatatctgccacatgtttgctaacaactttgcaggcaaaccatctctggcgagcacggccttgacggctccggtgtgtaagtacagcctgtatacacctcgaacgaacgacgaccatatggcattagaagttggaatggatctgacggcaaggatagttacaatggctcctccgatctccagctggagcgtatgaacgtctacttcaacgaggtgcgtacctcaaaatttcagcatctatgaaaacgctttgcaactcctgaccgcttctccaggccagcggaaacaagtatgtccctcgtgccgtcctcgttgatcttgagcctggtaccatggacgccgtccgtgccggtcccttcggtcagctcttccgtcccgacaacttcgttttcggccagtccggtgctggtaacaactgggccaagggtcactaaccttggagggtaacaaa

SEQ ID NO: 2 - DNA, 19 bp, synthetic construct
  source 1..19: mol_type = other DNA; note = ITS1 sequence
tccgtaggtgaacctgcgg

SEQ ID NO: 3 - DNA, 20 bp, synthetic construct
  source 1..20: mol_type = other DNA; note = ITS4 sequence
tcctccgcttattgatatgc

(19) United States
(12) Patent Application Publication
(10) Pub. No.: US 2025/0092352 A1
(43) Pub. Date: Mar. 20, 2025

(54) NON-TOXIN-PRODUCING ASPERGILLUS FLAVUS STRAIN, MICROBIAL INOCULANT, PREPARATION, AND USE

(71) Applicant: Xiangyang Academy of Agricultural Sciences, Xiangyang City (CN)

(72) Inventors: Dun WANG, Xiangyang City (CN); Xue WANG, Xiangyang City (CN); Peng FENG, Xiangyang City (CN); Xinsen RUAN, Xiangyang City (CN); Mengjie ZHU, Xiangyang City (CN); Jing DONG, Xiangyang City (CN); Qing YANG, Xiangyang City (CN); Shujuan ZHANG, Xiangyang City (CN)

(21) Appl. No.: 18/587,045

(22) Filed: Feb. 26, 2024

(30) Foreign Application Priority Data
Sep. 19, 2023 (CN) 202311211265.1

(51) Int. Cl. C12N 1/14; C12N 1/36; C12R 1/67

(52) U.S. Cl. CPC C12N 1/145; C12N 1/36; C12R 2001/67

(57) ABSTRACT

The present disclosure belongs to the technical field of microorganisms, and specifically relates to a non-toxin-producing Aspergillus flavus strain, a microbial inoculant, a preparation, and use. The present disclosure provides a non-toxin-producing Aspergillus flavus strain EXY1A109 with a deposit number of CCTCC No: M20221465. The strain can effectively inhibit the production of aflatoxin B₁, aflatoxin B₂, aflatoxin G₁, and aflatoxin G₂ by a toxin-producing Aspergillus flavus strain. The results of examples show that when an Aspergillus flavus strain EXY1A109 spore suspension and a strain CGMCC 3.4408 spore suspension are mixed in an equal volume, the Aspergillus flavus strain EXY1A109 spore suspension and the strain CGMCC 3.4408 spore suspension each have a concentration of 1×10⁶, and the strain EXY1A109 has a toxin-producing inhibition rate of 99.71% against the strain CGMCC 3.4408. This Aspergillus flavus strain shows a desirable application effect.

### CROSS REFERENCE TO RELATED APPLICATION

This patent application claims the benefit and priority of Chinese Patent Application No. 202311211265.1, filed with the China National Intellectual Property Administration on Sep. 19, 2023, the disclosure of which is incorporated by reference herein in its entirety as part of the present application.

### REFERENCE TO SEQUENCE LISTING

A computer readable XML file entitled “GWP20231209829”, created on Jan. 17, 2024, with a file size of about 4,629 bytes, contains the sequence listing for this application, has been filed with this application, and is hereby incorporated by reference in its entirety.

### TECHNICAL FIELD

The present disclosure belongs to the technical field of microorganisms, and specifically relates to a non-toxin-producing Aspergillus flavus strain, a microbial inoculant, a preparation, and use.

### BACKGROUND

At present, the production of aflatoxins is generally inhibited by chemical methods and physical methods. Their basic principle is to convert toxins into non-toxic compounds or complexes, or to degrade the toxins into non-toxic small molecule fragments, thereby achieving degrading the toxins. However, physical methods result in nutrient loss, while chemical methods are mostly reversible.

Related biodegradation methods have been studied at home and abroad to discuss biological control. Due to the large number of microorganisms in the soil, biocontrol bacteria used in biological control are ubiquitous in nature, including bacteria, yeasts, and algae.

Currently, non-toxin-producing strains of Aspergillus flavus have also been reported in the prior art, such as an Aspergillus flavus strain in Chinese patent CN103509723B. When a non-toxin-producing strain GZ-17 and a toxin-producing strain GD-1 are co-cultured at a spore concentration ratio of 10⁵:10⁵, the GZ-17 has a toxin-producing inhibition rate of 64.76% against the GD-1. When the non-toxin-producing strain GZ-17 and the toxin-producing strain GD-1 are co-cultured at a spore concentration ratio of 10⁶:10⁵ (a spore concentration of the non-toxin-producing strain GZ-17 is 10 times that of the toxin-producing strain GD-1), the GZ-17 has a toxin-producing inhibition rate of not less than 96.39% against the GD-1. However, the Aspergillus flavus strain GZ-17 still has a poor toxin-producing inhibition rate against aflatoxin-producing Aspergillus flavus strains.

### SUMMARY

An objective of the present disclosure is to provide a non-toxin-producing Aspergillus flavus strain EXY1A109. The strain EXY1A109 has a high toxin-producing inhibition rate against aflatoxin-producing Aspergillus flavus strains.

To solve the above technical problems, the present disclosure provides the following technical solutions:

The present disclosure provides a non-toxin-producing Aspergillus flavus strain EXY1A109 with a deposit number of CCTCC No: M20221465.

The present disclosure further provides a microbial inoculant including the non-toxin-producing Aspergillus flavus strain EXY1A109.

Preferably, the microbial inoculant is selected from the group consisting of an Aspergillus flavus strain EXY1A109 bacterial suspension, an Aspergillus flavus strain EXY1A109 fermentation broth, and the Aspergillus flavus strain EXY1A109.

The present disclosure further provides use of the Aspergillus flavus strain EXY1A109 or the microbial inoculant in production of a preparation for inhibiting toxin produced by an Aspergillus flavus strain.

Preferably, the Aspergillus flavus strain EXY1A109 in the preparation has a spore concentration of greater than or equal to 1×10⁵ CFU/mL.

The present disclosure further provides a preparation for inhibiting toxin produced by an Aspergillus flavus strain, including the Aspergillus flavus strain EXY1A109 and/or the microbial inoculant as an active ingredient.

Preferably, the preparation has a spore concentration of greater than or equal to 1×10⁵ CFU/mL.

Preferably, the toxin is one or more selected from the group consisting of aflatoxin B₁, aflatoxin B₂, aflatoxin G₁, and aflatoxin G₂.

The present disclosure further provides a method for inhibiting aflatoxin production, including mixing the Aspergillus flavus strain EXY1A109 with an aflatoxin-producing sample to allow symbiotic culture.

Preferably, the aflatoxin-producing sample is selected from the group consisting of a biological sample prepared from a toxin-producing Aspergillus flavus strain and a soil sample with the toxin-producing Aspergillus flavus strain.

The beneficial effects of the present disclosure are as follows: the present disclosure provides a non-toxin-producing Aspergillus flavus strain EXY1A109 with a deposit number of CCTCC No: M20221465. The strain can effectively inhibit the production of aflatoxin B₁, aflatoxin B₂, aflatoxin G₁, and aflatoxin G₂ by a toxin-producing Aspergillus flavus strain. The results of examples show that when an Aspergillus flavus strain EXY1A109 spore suspension and a standard toxin-producing Aspergillus flavus strain CGMCC 3.4408 spore suspension are mixed in an equal volume, the Aspergillus flavus strain EXY1A109 spore suspension and the standard toxin-producing Aspergillus flavus strain CGMCC 3.4408 spore suspension each have a concentration of 1×10⁵ CFU/mL, and the strain EXY1A109 has a toxin-producing inhibition rate of 92.94% against the strain CGMCC 3.4408. When the Aspergillus flavus strain EXY1A109 spore suspension and the strain CGMCC 3.4408 spore suspension are mixed in an equal volume, the Aspergillus flavus strain EXY1A109 spore suspension and the strain CGMCC 3.4408 spore suspension each have a concentration of 1×10⁶ CFU/mL, and the strain EXY1A109 has a toxin-producing inhibition rate of 99.71% against the strain CGMCC 3.4408. This strain is of great significance in inhibiting toxin-producing Aspergillus flavus from infecting agricultural products and reducing aflatoxin contamination in agricultural products.

### BRIEF DESCRIPTION OF THE DRAWINGS

To illustrate the examples of the present disclosure or the technical solutions in the prior art more clearly, the accompanying drawings required in the examples will be briefly introduced below.

FIGS. 1A-B show DG18 diagram and a control diagram of the antagonism experiment for toxin-producing strains and non-toxin-producing strains, where S represents SZ;

FIGS. 2A-B show colony appearance and conidium appearance of the Aspergillus flavus strain EXY1A109 on the DG18 medium, where a left picture represents a front side on the medium, and a right picture represents a back side on the medium;

FIGS. 3A-B show the back side for colonies of the Aspergillus flavus strain EXY1A109 on an AFPA medium;

FIG. 4 shows a spore appearance of the Aspergillus flavus strain EXY1A109 under a microscope (200×);

FIG. 5 shows the liquid-phase standard chromatograms of four aflatoxin mixed standards;

FIG. 6 shows the state of symbiotic mycelial balls of toxin-producing and non-toxin-producing strains; and

FIG. 7 shows a high-performance liquid chromatography (HPLC) chart illustrating an inhibitory effect of the strain.

### DETAILED DESCRIPTION OF THE EMBODIMENTS

The present disclosure provides a non-toxin-producing Aspergillus flavus strain EXY1A109 with a deposit number of CCTCC No: M20221465. The calmodulin of the Aspergillus flavus strain EXY1A109 has a gene sequence shown in SEQ ID NO: 1.

The currently developed bacterial biocontrol bacteria, such as lactic acid bacteria and bacilli, can reduce aflatoxin pollution, but are easily affected by the soil environment in different regions, showing unsatisfactory application results.

Therefore, it is necessary to select a non-toxin-producing Aspergillus flavus strain with a high inhibitory ability from the soil. This strain competitively inhibits the growth of toxin-producing Aspergillus flavus in the field, regulates and controls the growth of high-producing virulent Aspergillus flavus in the soil, and prevents aflatoxin contamination of different grain or oil crops, showing desirable application prospects. A non-toxin-producing Aspergillus flavus strain EXY1A109 is isolated from soil samples in the main peanut-producing area of Xiangyang City. This strain exhibits high adaptability to the soil environment and is used to inhibit the growth of toxin-producing Aspergillus flavus in the field.

In the present disclosure, the Aspergillus flavus strain EXY1A109 has a colony appearance that is dense and filamentous, with many conidial structures and a spore size of 200 μm to 500 μm in diameter.

In the present disclosure, the calmodulin sequence of EXY1A109 is compared with the data in the NCBI database to preliminarily determine the classification information of the strain. The identification results show that the strain EXY1A109 is an Aspergillus flavus strain.

The present disclosure further provides a microbial inoculant including the non-toxin-producing Aspergillus flavus strain EXY1A109.

In the present disclosure, the microbial inoculant is preferably selected from the group consisting of an Aspergillus flavus strain EXY1A109 bacterial suspension and an Aspergillus flavus strain EXY1A109 fermentation broth.

In the present disclosure, a preparation method of the Aspergillus flavus strain EXY1A109 bacterial suspension preferably includes: inoculating the Aspergillus flavus strain EXY1A109 on a DG18 medium to allow culture to obtain spores, and eluting the spores with Tween 80 with a mass concentration of 0.1% to obtain an Aspergillus flavus strain conidium suspension. The spores are counted using an optical microscope, preferably adjusted to a spore concentration of greater than or equal to 1×10⁵ CFU/mL, and then stored in a 4° C. refrigerator for later use.

In the present disclosure, a preparation method of the Aspergillus flavus strain EXY1A109 fermentation broth preferably includes: inoculating 1 mL of an Aspergillus flavus strain bacterial solution into 30 mL of a liquid Sabouraud medium, and conducting symbiotic culture in a dark shaker at 28° C. and 200 rpm for 7 d. The Aspergillus flavus strain bacterial solution has a spore concentration of preferably greater than or equal to 1×10⁵ CFU/mL.

The present disclosure further provides use of the Aspergillus flavus strain EXY1A109 or the microbial inoculant in production of a preparation for inhibiting toxin produced by an Aspergillus flavus strain.

In the present disclosure, the Aspergillus flavus strain EXY1A109 in the preparation has a spore concentration of greater than or equal to 1×10⁵ CFU/mL, more preferably greater than or equal to 4×10⁵ CFU/mL, and even more preferably 1×10⁶ CFU/mL.

The preparation preferably further includes conventional preparation components, which are not specifically limited.

The present disclosure further provides a preparation for inhibiting toxin produced by an Aspergillus flavus strain, including the Aspergillus flavus strain EXY1A109 and/or the microbial inoculant as an active ingredient.

In the present disclosure, the preparation has a working temperature of preferably 25° C. to 30° C., more preferably 26° C. to 19° C., and even more preferably 28° C.

In the present disclosure, the Aspergillus flavus strain EXY1A109 in the preparation has a spore concentration of preferably greater than or equal to 1×10⁵ CFU/mL, more preferably 1×10⁶ CFU/mL.

In the present disclosure, the toxins inhibited by the Aspergillus flavus strain EXY1A109 preferably include one or more of aflatoxin B₁, aflatoxin B₂, aflatoxin G₁, and aflatoxin G₂, more preferably the aflatoxin B₁, the aflatoxin B₂, the aflatoxin G₁, and the aflatoxin G₂.

The present disclosure further provides a method for inhibiting aflatoxin production, including mixing the Aspergillus flavus strain EXY1A109 with an aflatoxin-producing sample to allow symbiotic culture. The aflatoxin-producing sample is preferably selected from the group consisting of a biological sample prepared from a toxin-producing Aspergillus flavus strain and a soil sample with the toxin-producing Aspergillus flavus strain. The biological sample preferably includes a spore suspension. There is no special limitation on a mixing method, and conventional methods can be used.

In the present disclosure, preferably the Aspergillus flavus strain EXY1A109 and the biological sample prepared by the toxin-producing Aspergillus flavus strain is mixed to allow symbiotic culture; more preferably, the Aspergillus flavus strain EXY1A109 and the spore suspension prepared by the toxin-producing Aspergillus flavus strain is mixed to allow symbiotic culture; and even more preferably, the Aspergillus flavus strain EXY1A109 spore suspension and the spore suspension prepared by the toxin-producing Aspergillus flavus strain is mixed to allow symbiotic culture.

In the present disclosure, the Aspergillus flavus strain EXY1A109 spore suspension is preferably mixed with the spore suspension prepared from the toxin-producing Aspergillus flavus strain according to equal spore concentrations and equal volumes.

In the present disclosure, the symbiotic culture preferably includes inoculating the Aspergillus flavus strain EXY1A109 spore suspension and the spore suspension prepared from the toxin-producing Aspergillus flavus strain into a medium to allow the symbiotic culture. The Aspergillus flavus strain EXY1A109 spore suspension has a spore concentration of preferably greater than or equal to 1×10⁵ CFU/mL, more preferably 1×10⁶ CFU/mL. The spore suspension prepared from the aflatoxin-producing Aspergillus flavus strain has a spore concentration of preferably greater than or equal to 1×10⁵ CFU/mL, more preferably 1×10⁶ CFU/mL.

In the present disclosure, the symbiotic culture is conducted at preferably 25° C. to 30° C., more preferably 28° C. for preferably 6 d to 8 d, more preferably 7 d under preferably 180 r/min to 220 r/min, more preferably 200 r/min. During the inoculation, the Aspergillus flavus strain EXY1A109 spore suspension, the spore suspension prepared from the aflatoxin-producing Aspergillus flavus strain, and the medium are at a volume ratio of preferably (1-2):(1-2):30, more preferably 1:1:30.

In the present disclosure, a medium used in the symbiotic culture is preferably a liquid Sabouraud medium. The liquid Sabouraud medium is preferably a commercially available product. In an example, the liquid Sabouraud medium is preferably purchased from Qingdao Hi-Tech Industrial Park Hope Bio-Technology Co., Ltd.

In the present disclosure, the non-toxin-producing Aspergillus flavus strain EXY1A109 can inhibit the production of aflatoxin by any toxin-producing Aspergillus flavus strain. In an example, the aflatoxin-producing Aspergillus flavus strain is a standard toxin-producing Aspergillus flavus strain CGMCC3.4408. When an Aspergillus flavus strain EXY1A109 spore suspension and a standard toxin-producing Aspergillus flavus strain CGMCC 3.4408 spore suspension are mixed in an equal volume, the Aspergillus flavus strain EXY1A109 spore suspension and the standard toxin-producing Aspergillus flavus strain CGMCC 3.4408 spore suspension each have a concentration of 1×10⁵ CFU/mL, and the strain EXY1A109 has a toxin-producing inhibition rate of 92.94% against the strain CGMCC 3.4408. When the Aspergillus flavus strain EXY1A109 spore suspension and the strain CGMCC 3.4408 spore suspension are mixed in an equal volume, the Aspergillus flavus strain EXY1A109 spore suspension and the strain CGMCC 3.4408 spore suspension each have a concentration of 1×10⁶ CFU/mL, and the strain EXY1A109 has a toxin-producing inhibition rate of 99.71% against the strain CGMCC 3.4408.

The present disclosure provides a method for inhibiting aflatoxin production, including mixing the Aspergillus flavus strain EXY1A109 with a soil sample with a toxin-producing Aspergillus flavus strain, and then conducting symbiotic culture. More preferably, the Aspergillus flavus strain EXY1A109 is prepared into a bacterial suspension or a fermentation broth and then mixed with the soil sample with a toxin-producing Aspergillus flavus strain to allow the symbiotic culture. The spore concentration of the Aspergillus flavus strain EXY1A109 bacterial suspension or fermentation broth is preferably greater than or equal to 1×10⁵ CFU/mL, more preferably 1×10⁶ CFU/mL.

In the present disclosure, the symbiotic culture is conducted at preferably 25° C. to 37° C., more preferably 29° C. to 32° C., and even more preferably 28° C. The symbiotic culture is conducted for preferably 2 d to 15 d, more preferably 5 d to 7 d, and more preferably 7 d.

In the present disclosure, the aflatoxin produced by Aspergillus flavus in the soil sample has a mass concentration of preferably less than or equal to 1,077 μg/kg. The aflatoxin mass concentration of 1,077 μg/kg refers to a total concentration of the aflatoxins G₂, G₁, B₂, and B₁.

The present disclosure provides a non-toxin-producing Aspergillus flavus strain, which is Aspergillus flavus EXY1A109 with a deposit number of CCTCC No: M20221465 in the China Center for Type Culture Collection. Experiments have proven that the strain of the present disclosure has an inhibitory effect on the toxin production of aflatoxin-producing Aspergillus flavus strain, and when a spore concentration ratio of the non-toxin-producing strain EXY1A109 to the standard toxin-producing strain CGMCC 3.4408 is 1×10⁶:1×10⁶, the toxin-producing inhibition rate of the non-toxin-producing strain against toxin-producing strain reaches 99.71%. This strain shows a great application potential in inhibiting the growth of toxin-producing Aspergillus flavus strain and inhibiting the production of aflatoxin metabolites. This strain is of great significance in inhibiting toxin-producing Aspergillus flavus strains from infecting agricultural products and reducing aflatoxin contamination in agricultural products.

In order to further illustrate the present disclosure, the technical solutions provided by the present disclosure are described in detail below in connection with accompanying drawings and examples, but these examples should not be understood as limiting the claimed scope of the present disclosure.

### Culture Used in the Examples

DG18 medium is purchased from Qingdao Hi-Tech Industrial Park Hope Bio-Technology Co., Ltd.;
AFPA medium is purchased from Qingdao Hi-Tech Industrial Park Hope Bio-Technology Co., Ltd.; and
liquid Sabouraud medium is purchased from Qingdao Hi-Tech Industrial Park Hope Bio-Technology Co., Ltd.

### EXAMPLE 1

### 1. Collection and Processing of Soil Sample

The soil in the main peanut producing area of Xiangyang City was divided into three categories: sandy loam, gravel soil, and yellow clay. During the peanut maturity period, more than 20 soil samples were taken from the main peanut producing areas in 10 counties and cities in Xiangyang City, including Xiangzhou, Zaoyang, Yicheng, Gucheng, Nanzhang, and High-tech Zone, and were temporarily stored in a low-temperature refrigerator at 4° C. for later use. Samples were obtained after grinding the soil, 10.0 g of each sample was added into an Erlenmeyer flask, added with 90 mL of sterile water, and mixed in a shaker for 5 min to obtain 100 mL of a sample basic solution for later use.

2. Isolated Culture of Bacterial Strain

100 μL of the basic solution was coated evenly on a DG18 medium plate with a sterilized coating rod, repeated 4 times, and cultured at 28° C. in the dark for 5 d. The yellow-green bacterial plaques were selected and inoculated on an AFPA medium, and subjected to isolated culture under the same conditions for 3 d to ensure that a single colony grew on one plate. More than 200 strains were isolated and purified from 20 soil samples through colony morphology identification.

### 3. Identification of Bacterial Species

After the selected strains were cultured on AFPA medium and colonies grew, the plaques with white front and orange back were initially identified as Aspergillus flavus. The Aspergillus flavus strain was then transferred to DG18 medium, subjected to isolated culture at 28° C.±1° C. in the dark for 5 d, separated and purified until a single colony grew, and the single colony was sequenced and identified. A genomic DNA was crudely extracted, the corresponding primers were selected to amplify the specific fragment, and the DNA was sent to Beijing Qingke Biotechnology Co., Ltd. Wuhan Branch for sequencing and identification. The sequencing results were compared for homology using BLAST software. All strains used in the comparison experiment were identified as Aspergillus flavus strains, and their homology with Aspergillus flavus was over 98%.

### EXAMPLE 2 SCREENING OF NON-TOXIN-PRODUCING STRAIN

### 1. Screening of Non-Toxin-Producing Strain

The strains isolated from the soil of the main peanut producing area of Xiangyang in Example 1 were screened, and liquid-phase fluorescence test was conducted to detect the toxin-producing strain (GB5009.22-2016). The results showed that Aspergillus flavus in the soil of peanut production areas in different areas of Xiangyang mainly produced AFB 1 (aflatoxin B₁). The AFB1>AFB2 (aflatoxin B₂)>AFG2 (aflatoxin G₂)>AFG1 (aflatoxin G₁), which also caused greater harm to the contamination of peanut fruits in the later period. The highest toxin-producing strain produced by the highly toxin-producing strain reached a maximum AFB1 content of 1,407.9 μg/kg per gram of soil, indicating that peanut cultivation in this area had a greater risk of contamination. 49 non-toxin-producing strains were selected, two of which were named ZY11 and YC10.

### 2 Preliminary Screening of Antagonistic Properties of Non-Toxin-Producing Strain YC10

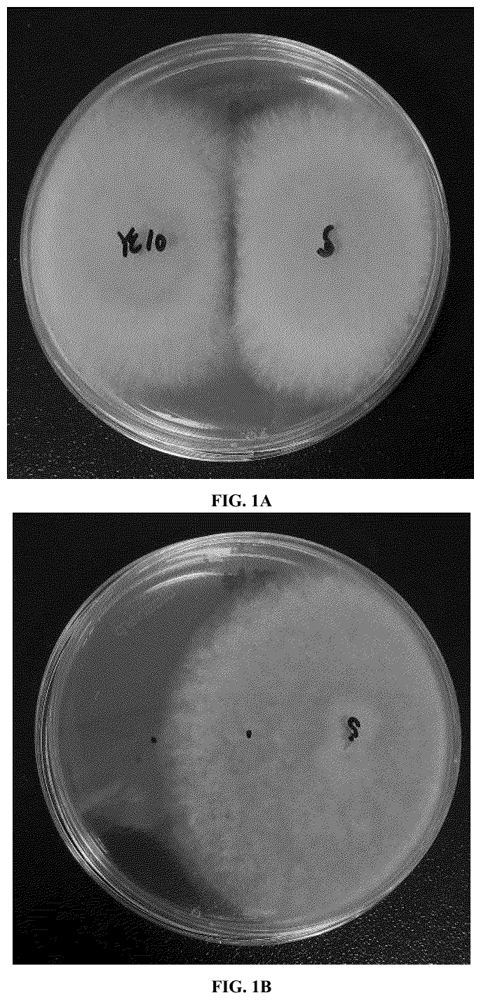

The screened non-toxin-producing strain YC10 was co-cultured with a standard toxin-producing Aspergillus flavus strain CGMCC 3.4408 (referred to as SZ) on a plate, and 4 parallel plates were set up. The strain YC10 was found to have an obvious inhibition zone, and the inhibition zone exceeded 0.5 cm, as shown in FIGS. 1A-B; where a left picture represented the antagonism diagram of YC10 and SZ, and a right picture represented the control diagram of SZ alone.

After continuing to observe the symbiotic characteristics of strains YC10 and CGMCC 3.4408 under UV light, it was found that strain YC10 had an obvious inhibitory effect on the toxin-producing strain CGMCC 3.4408, and the aflatoxin fluorescent spot was significantly weakened under fluorescence.

According to FIGS. 1A-B, the strain YC10 had an inhibitory effect on toxin production by CGMCC 3.4408.

A genomic DNA of the strain YC10 was crudely extracted, and corresponding primers were selected to amplify the specific fragment. The primers used in PCR amplification were: ITS1 sequence 5′-TCCGTAGGTGAACCTGCGG-3′ (SEQ ID NO: 2); ITS4 sequence 5′-TCCTCCGCTTATTGATATGC-3′ (SEQ ID NO: 3). The composition of the PCR system was shown in Table 1.

The PCR program was shown in Table 2: 98° C. for 10 s; 58° C. for 10 s; 72° C. for 20 s, 35 cycles.

The PCR amplification product was sent to Beijing Qingke Biotechnology Co., Ltd. Wuhan Branch for sequencing and identification. The sequencing results were compared for homology with Aspergillus flavus using BLAST software, and the homology with Aspergillus flavus was 99.80%.

TABLE 1
PCR system composition

| | Volume (μL) |
|---|---|
| PCR Mix | 25 |
| ITS1(10 μM) | 2 |
| ITS4(10 μM) | 2 |
| YC10 genomic DNA as template | 1 |

ddH₂O making up to 20 mL

TABLE 2
PCR program

| Temperature ° C. | Time | Number of cycles |
|---|---|---|
| 98 | 2 min | |
| 98 | 10 s | 35 |
| 58 | 10 s | |
| 72 | 20 s | |
| 72 | 5 min | |

The PCR amplification and sequencing results of calmodulin of strain YC10 were shown in SEQ ID NO: 1.

SEQ ID NO: 1:
GCGGGATCTATGCCTTCGAGTTAGTATGCTTTGGACCAAGGAACTCC
TCAAAAGCATGATCTCGGATGTGTCCTGTTATATCTGCCACATGTTT
GCTAACAACTTTGCAGGCAAACCATCTCTGGCGAGCACGGCCTTGAC
GGCTCCGGTGTGTAAGTACAGCCTGTATACACCTCGAACGAACGACG
ACCATATGGCATTAGAAGTTGGAATGGATCTGACGGCAAGGATAGTT
ACAATGGCTCCTCCGATCTCCAGCTGGAGCGTATGAACGTCTACTTC
AACGAGGTGCGTACCTCAAAATTTCAGCATCTATGAAAACGCTTTGC
AACTCCTGACCGCTTCTCCAGGCCAGCGGAAACAAGTATGTCCCTCG
TGCCGTCCTCGTTGATCTTGAGCCTGGTACCATGGACGCCGTCCGTG
CCGGTCCCTTCGGTCAGCTCTTCCGTCCCGACAACTTCGTTTTCGGC
CAGTCCGGTGCTGGTAACAACTGGGCCAAGGGTCACTAACCTTGGAG
GGTAACAAA.

The strain YC10 was named Aspergillus flavus strain EXY1A109 during biological preservation.

### 3. Morphological Identification of Strain

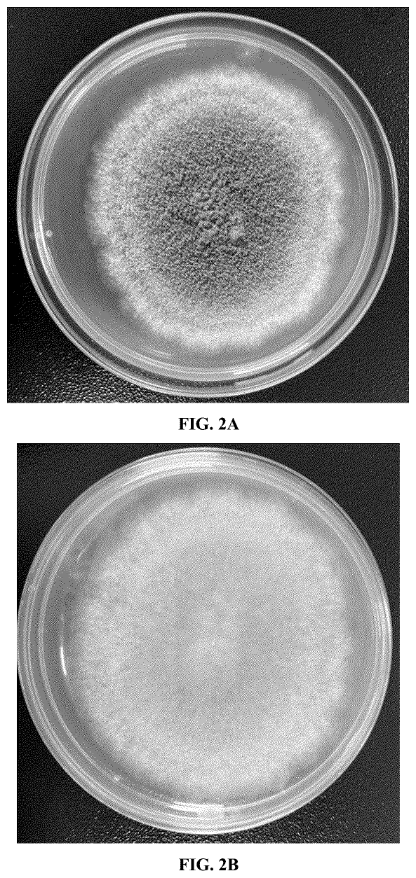

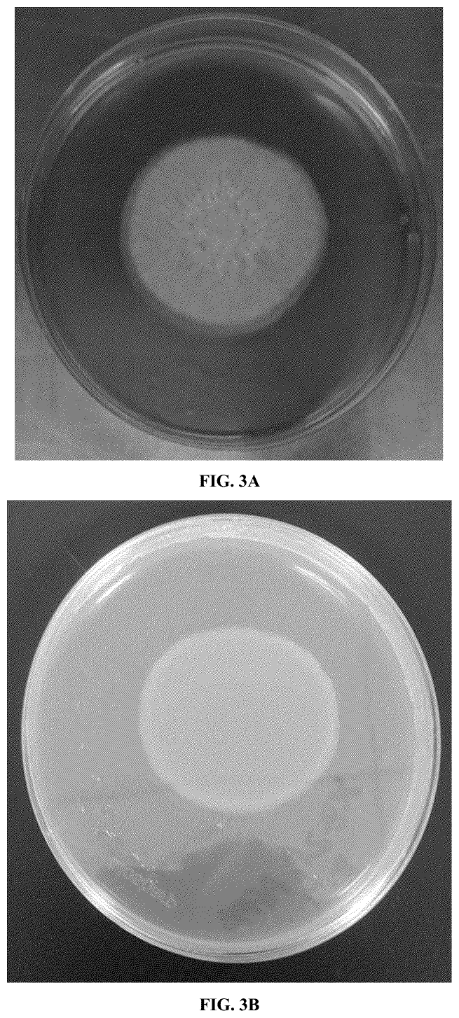

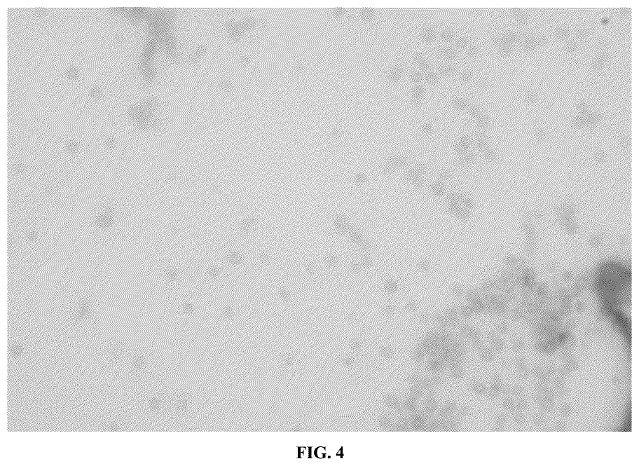

The colony appearance and conidium appearance of Aspergillus flavus strain EXY1A109 on DG18 medium were shown in FIGS. 2A-B; the diagram of the back side of the colony of Aspergillus flavus strain EXY1A109 on AFPA medium was shown in FIGS. 3A-B, where two pictures in FIGS. 3A-B represented the front and back views of the same culture time. The spore appearance of Aspergillus flavus strain EXY1A109 under the microscope (200×) was shown in FIG. 4.

### EXAMPLE 3 DETERMINATION OF THE TOXIN-PRODUCING INHIBITION RATE OF STRAIN

### 1. Plotting of Standard Curve

Aflatoxin B₁, aflatoxin B₂, aflatoxin G₁, and aflatoxin G₂ mixed standards were used as standards for HPLC tandem-column-post and photochemical derivatization detection.

The HPLC tandem-column-post photochemical derivatization method had the following parameters:

The chromatographic conditions included: the chromatographic column was C18 (5 μm, 4.6 mm×150 mm), the column temperature was 35° C.; the mobile phase was methanol: water (45:55, V:V); the flow rate was 0.9 mL/min; detection conditions were: photochemistry derivatizer 254 nm; detection with fluorescence detector, excitation wavelength 360 nm, emission wavelength 440 nm, injection volume 10 μL.

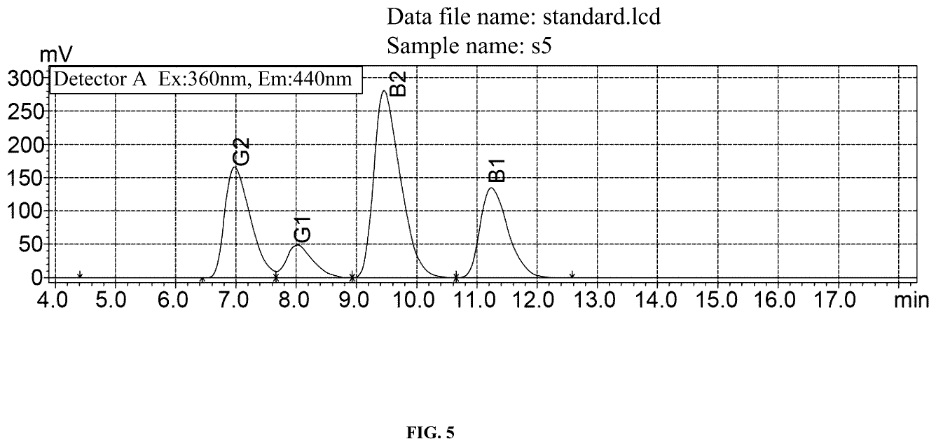

The results were shown in FIG. 5. As shown in FIG. 5, the peak times of the aflatoxin (AFT) mixed standards were aflatoxin B₁, aflatoxin B₂, aflatoxin G₁, and aflatoxin G₂, with desirable separation. Among the aflatoxin standards, the peak time of AFB1 was 11.3 min, the peak time of AFB2 was 9.5 min, the peak time of AFG1 was 8 min, and the peak time of AFG2 was 7 min. The peak elution time of the four substances was 13 min. A standard curve was plotted based on the chromatographic area (Y) and concentration (X, μg/L) to obtain a linear regression equation (Table 3). There was desirable linear relationship between the 4 standards.

TABLE 3
Standard curve of aflatoxins

| | | Correlation | Limit of | Limit of |
|---|---|---|---|---|
| Aflatoxin | Standard | R2 | detection | quantitation |
| standards | curve | coefficient | (μg/kg) | (μg/kg) |
| AFG2 | Y = 39296.0X- | 0.9999685 | 0.26 | 1.3 |
| | 17563.0 | | | |
| AFG1 | Y = 12069.0X- | 0.9999565 | 0.36 | 1.3 |
| | 9167.47 | | | |
| AFB2 | Y = 88965.4X- | 0.9999728 | 0.18 | 1.0 |
| | 57284.3 | | | |
| AFB1 | Y = 44595.7X- | 0.9999687 | 0.24 | 1.0 |
| | 35890.3 | | | |

### 2. Toxin-Producing Inhibition Rate

Aspergillus flavus strains EXY1A109 and ZY11 among the selected non-toxin-producing Aspergillus flavus strains were subjected to symbiotic culture with the standard toxin-producing Aspergillus flavus strain CGMCC 3.4408 (referred to as SZ) according to Treatments 1 to 3. The standard toxin-producing Aspergillus flavus strain CGMCC 3.4408 was purchased from the Oil Plant Research Institute of the Chinese Academy of Agricultural Sciences.

The single colony of Aspergillus flavus strain EXY1A109 was inoculated on the DG18 medium, and the obtained spores were eluted with a Tween 80 aqueous solution with a mass concentration of 0.1% to prepare a spore suspension.

The single colony of CGMCC 3.4408 was inoculated on the DG18 medium, and the obtained spores were cluted with a Tween 80 aqucous solution with a mass concentration of 0.1% to prepare a spore suspension.

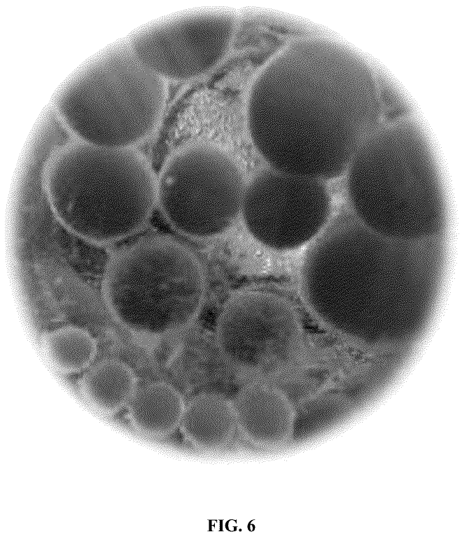

Treatment 1: symbiotic culture: the Aspergillus flavus strain EXY1A109 and strain CGMCC 3.4408 were accurately counted to determine a number of spores under a microscope, the concentration of the 2 spore suspensions were adjusted to 10⁵ CFU/mL, 1 mL of non-toxin-producing spore suspension and 1 mL of spore suspension of strain CGMCC3.4408 was inoculated into 30 mL of liquid Sabouraud medium, and subjected to symbiotic culture on a dark shaker at 28° C. and 200 r/min for 7 d. The growth status was observed every day to obtain a symbiotic culture solution. When the Aspergillus flavus strain EXY1A109 and the strain CGMCC 3.4408 (referred to as SZ) were subjected to symbiotic culture at a spore concentration of 10⁵:10⁵, a symbiotic culture status was shown in FIG. 6, indicating that the growth of mycelial balls of CGMCC 3.4408 was significantly inhibited.

Treatment 2: the concentrations of the 2 spore suspensions were adjusted to 10⁶ CFU/mL, and the Aspergillus flavus strain EXY1A109 and strain CGMCC3.4408 were subjected to symbiotic culture. When the Aspergillus flavus strain EXY1A109 and standard toxin-producing Aspergillus flavus strain CGMCC 3.4408 (referred to as SZ) were subjected to symbiotic culture at a spore concentration of 10⁶:10⁶, the other conditions were the same as those in Treatment 1.

Treatment 3: the conditions for symbiotic culture of ZY11 and the standard toxin-producing Aspergillus flavus strain CGMCC 3.4408 (referred to as SZ) were the same as those in Treatment 1.

The culture solutions obtained from Treatments 1 to 3 were subjected to cell disruption and then filtered to obtain a crude extract separately, without cross-contamination. 1 mL of the crude extract was passed through an immunoaffinity column, and eluted with 1 mL of methanol. A resulting eluate was collected and tested for AFT toxicity.

The aflatoxin B₁, aflatoxin B₂, aflatoxin G₁, and aflatoxin G₂ mixed standards in step 1 were diluted with methanol to prepare aflatoxin (AFT) standard solutions with 6 concentration gradients of 1, 5, 10, 50, 100, and 500 μg/L. The HPLC tandem-column-post photochemical derivatization was conducted to determine aflatoxins in fermentation broth.

The chromatographic conditions were as follows: the chromatographic column was C18 (5 μm, 4.6 mm×150 mm), the column temperature was 35° C.; the mobile phase was methanol: water (45:55, V:V); the flow rate was 0.9 mL/min; detection conditions were: photochemistry derivatizer 254 nm; detection with fluorescence detector, excitation wavelength 360 nm, emission wavelength 440 nm, injection volume 10 μL.

After HPLC-post-column photochemical derivatization detection, the average value of 3 parallel results was shown in Table 4. The average AFT value of the standard toxin-producing Aspergillus flavus strain CGMCC 3.4408 was 1,072.95 μg/g. When the spore concentration was 1×10⁵ CFU/mL, the non-toxin-producing Aspergillus flavus strain EXY1A109 inhibited the toxin production of strain CGMCC 3.4408 by 92.94%; when the spore concentration was 1×10⁶ CFU/mL, the non-toxin-producing Aspergillus flavus strain EXY1A109 inhibited the toxin production of strain CGMCC 3.4408 by 99.71%. One possibility for the antibacterial effect was that non-toxin-producing strains competitively inhibited the growth of toxin-producing strains, while another possibility was that some biological enzymes were produced during the liquid fermentation to inhibit the production of aflatoxins.

TABLE 4
Inhibitory effects of selected Aspergillus flavus
strain on toxin-producing strains

| | | Inhibited | Toxin- |
|---|---|---|---|
| | Toxin | toxin | producing |
| | produced | produced | inhibition |
| Strain No. | (μg/kg) | (μg/kg) | rate % |
| CGMCC3.4408(SZ)(10⁵) | 1072.95 ± 1.67 | | |
| EXY1A109:SZ(10⁵:10⁵) | 75.77 ± 1.35 | 997.19 ± 1.35 | 92.94 ± 0.67b |
| EXY1A109:SZ(10⁶:10⁶) | 3.11 ± 0.62 | 1069.83 ± 5.03 | 99.71 ± 0.26a |
| ZY11:SZ(10⁵:10⁵) | 1053.58 ± 5.04 | 19.37 ± 5.04 | 1.81 ± 2.33a |

Note:
different letters in Table 4 represented significant differences at the P < 0.05 level.
3. HPLC chart of an inhibitory effect of the strain

(1) Symbiotic culture: the non-toxin-producing strain EXY1A109 and strain CGMCC 3.4408 were accurately counted to determine a number of spores under a microscope, the concentration of the 2 spore suspensions were adjusted to 10⁵ CFU/mL, 1 mL of strain EXY1A109 spore suspension and 1 mL of strain CGMCC3.4408 spore suspension were inoculated into 30 mL of liquid Sabouraud medium, and subjected to symbiotic culture on a dark shaker at 28° C. and 200 r/min for 7 d. The growth status was observed every day to obtain a symbiotic culture solution.

1 mL of strain CGMCC 3.4408 spore suspension with a spore concentration of 10⁵ CFU/mL and 1 mL of sterile water were inoculated into 30 mL of liquid Sabouraud medium as a blank control to obtain a control culture solution.

(2) Extraction and purification: the symbiotic culture solution and the control culture solution were subjected to cell disruption, and filtered with sterile gauze while avoiding cross-contamination. After centrifugation to obtain a supernatant, 1 mL of the supernatant was purificd through an immunoaffinity column, cluted with 2 mL of methanol to replace the solvent, and filtered through a 0.22 μm microporous membrane. 1 mL of a sample was collected for liquid chromatography detection, and the HPLC tandem-column-post photochemical derivatization method was the same as above.

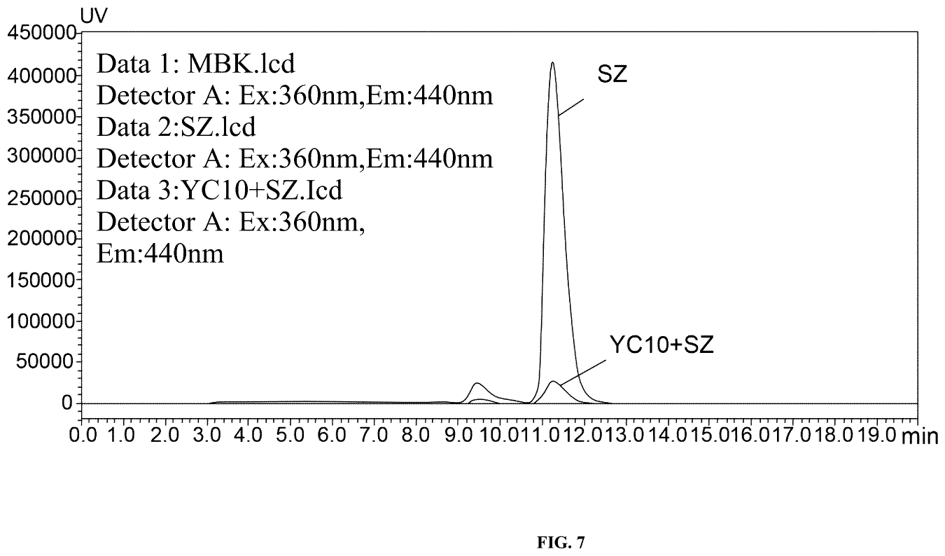

The HPLC chart of Aspergillus flavus strain EXY1A109 was shown in FIG. 7. According to FIG. 7, aflatoxin in the co-culture group showed low production. In FIG. 7, the SZ line (large peak area) represented the control group and the YC10+SZ line (small peak area) represented the co-culture group.

In summary, the Aspergillus flavus strain EXY1A109 of the present disclosure has an inhibitory effect on the toxin production of aflatoxin-producing Aspergillus flavus strain, and when a spore concentration ratio of the Aspergillus flavus strain EXY1A109 to the standard toxin-producing strain CGMCC 3.4408 is 1×10⁶:1×10⁶, the toxin-producing inhibition rate of the non-toxin-producing strain against toxin-producing strain reaches 99.71%. This strain shows a great application potential in inhibiting the growth of toxin-producing Aspergillus flavus strain and inhibiting the production of aflatoxin metabolites. This strain is of great significance in inhibiting toxin-producing Aspergillus flavus strains from infecting agricultural products and reducing aflatoxin contamination in agricultural products.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A microbial inoculant, comprising a non-toxin-producing Aspergillus flavus strain EXY1A109 with a deposit number of CCTCC No: M20221465.

2. The microbial inoculant according to claim 1, wherein the microbial inoculant is selected from the group consisting of an Aspergillus flavus strain EXY1A109 bacterial suspension, an Aspergillus flavus strain EXY1A109 fermentation broth, and the Aspergillus flavus strain EXY1A109.

3. A method for producing a preparation for inhibiting toxin produced by an Aspergillus flavus strain, comprising applying the microbial inoculant according to claim 1.

4. A method for producing a preparation for inhibiting toxin produced by an Aspergillus flavus strain, comprising applying the microbial inoculant according to claim 2.

5. The method according to claim 3, wherein the Aspergillus flavus strain EXY1A109 in the preparation has a spore concentration of greater than or equal to 1×10⁵ CFU/mL.

6. The method according to claim 4, wherein the Aspergillus flavus strain EXY1A109 in the preparation has a spore concentration of greater than or equal to 1×10⁵ CFU/mL.

7. A preparation for inhibiting toxin produced by an Aspergillus flavus strain, comprising the microbial inoculant according to claim 1 as an active ingredient.

8. A preparation for inhibiting toxin produced by an Aspergillus flavus strain, comprising the microbial inoculant according to claim 2 as an active ingredient.

9. The preparation according to claim 7, wherein the preparation has a spore concentration of greater than or equal to 1×10⁵ CFU/mL.

10. The preparation according to claim 8, wherein the preparation has a spore concentration of greater than or equal to 1×10⁵ CFU/mL.

11. The preparation according to claim 7, wherein the toxin is one or more selected from the group consisting of aflatoxin B₁, aflatoxin B₂, aflatoxin G₁, and aflatoxin G₂.

12. The preparation according to claim 8, wherein the toxin is one or more selected from the group consisting of aflatoxin B₁, aflatoxin B₂, aflatoxin G₁, and aflatoxin G₂.

13. A method for inhibiting aflatoxin production, comprising mixing the Aspergillus flavus strain EXY1A109 according to claim 1 with an aflatoxin-producing sample to allow symbiotic culture.

14. The method according to claim 13, wherein the aflatoxin-producing sample is selected from the group consisting of a biological sample prepared from a toxin-producing Aspergillus flavus strain and a soil sample with the toxin-producing Aspergillus flavus strain.
